(19) United States
(12) Patent Application Publication
(10) Pub. No.: US 2024/0268882 A1
(43) Pub. Date: Aug. 15, 2024

(54) ELECTROSURGICAL FORCEPS WITH TISSUE CONTACT SENSING

(71) Applicant: Covidien LP, Mansfield, MA (US)

(72) Inventors: James D. Allen, IV, Broomfield, CO (US); Christopher A. Valentine, Longmont, CO (US); Grant T. Sims, Lakewood, CO (US)

(21) Appl. No.: 18/565,011

(22) PCT Filed: May 10, 2022
(86) PCT No.: PCT/IB2022/054340

§ 371 Date: Nov. 28, 2023

Related U.S. Application Data
(60) Provisional application No. 63/194,521, filed on May 28, 2021.

(51) Int. Cl. A61B 18/14; A61B 18/00; A61B 18/12; A61B 34/35

(52) U.S. Cl. CPC A61B 18/1445; A61B 18/1206; A61B 34/35; A61B 2018/0063; A61B 2018/00642; A61B 2018/00708; A61B 2018/0072; A61B 2018/00761; A61B 2018/00767; A61B 2018/0091; A61B 2018/1253; A61B 2018/1452

(57) ABSTRACT

A surgical instrument includes a housing having an elongated shaft extending distally therefrom and configured to support an end effector assembly at a distal end thereof. The end effector assembly includes first and second jaw members each having a tissue sealing plate disposed thereon and adapted to connect to an electrosurgical energy source for delivery thereto upon activation thereof. A sensor is disposed one or both of the tissue sealing plates of the first and second jaw members and is configured to communicate data relating to a location of tissue disposed between the first and second jaw members to the electrosurgical energy source. The electrosurgical energy source, in turn, uses the tissue location data to adjust one or more parameters associated with the delivery of electrosurgical energy to the tissue upon activation thereof.

### 1. TECHNICAL FIELD

The present disclosure relates generally to the field of surgical instruments. In particular, the disclosure relates to electrosurgical forceps that is configured to identify the location of the tissue disposed between a pair of jaw members of the surgical instrument prior to or during activation.

### 2. BACKGROUND OF RELATED ART

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue. A detailed discussion of the use of an electrosurgical forceps may be found in U.S. Pat. No. 7,255,697 to Dycus et al.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To effect a proper seal, particularly in relatively large vessels, two predominant mechanical parameters must be accurately controlled; the pressure applied to the vessel, and the gap distance established between the electrodes.

Both the pressure and gap distance influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied the tissue may have a tendency to move before an adequate seal can be generated. The thickness of a typical effective tissue seal is optimally between about 0.001 and about 0.006 inches. Below this range, the seal may shred or tear and above this range the vessel walls may not be effectively joined. Closure pressures for sealing large tissue structures preferably fall within the range of about 3 kg/cm² to about 16 kg/cm².

Many endoscopic surgical instruments utilize handle or levers to actuate the end effector assembly typically disposed at a distal end of the instrument. For example, actuation of the handle correspondingly actuates the jaw members. Once closed about tissue electrical energy is delivered to treat tissue. With open forceps, two opposing handles are pivotable relative to tone another to grasp tissue prior to energizing the jaw members.

In some instances, it may be beneficial for the surgeon to identify the position of the tissue disposed between the jaw members for configuring the amount or type of electrosurgical energy coming from the generator upon activation or defaulting to a tissue treatment type. In other instances, detecting the location of the tissue in the jaw members may be used as a safety feature.

### SUMMARY

As used herein, the term “distal” refers to the portion of the instrument or component thereof that is being described that is further from a user, while the term “proximal” refers to the portion of the instrument or component thereof that is being described that is closer to a user. Further, to the extent consistent, any of the aspects described herein may be used in conjunction with any of the other aspects described herein. As used herein the term “tissue” is meant to include variously-sized vessels.

Provided in accordance with aspects of the present disclosure is a surgical instrument including a housing having an elongated shaft extending distally therefrom and configured to support an end effector assembly at a distal end thereof. The end effector assembly includes first and second jaw members each having a tissue sealing plate disposed thereon and adapted to connect to an electrosurgical energy source for delivery thereto upon activation thereof. A sensor is disposed on one or both of the tissue sealing plates of the first and second jaw members and is configured to communicate data relating to a location of tissue disposed between the first and second jaw members to the electrosurgical energy source. The electrosurgical energy source, in turn, uses the tissue location data to adjust one or more parameters associated with the delivery of electrosurgical energy to the tissue upon activation thereof.

In aspects according to the present disclosure, the one or more parameters associated with the delivery of electrosurgical energy to tissue includes power, current, voltage, duration, and/or pulse width. In other aspects according to the present disclosure, the surgical instrument includes a plurality of sensors disposed along one or both tissue sealing plates, the plurality of sensors communicating data relating to the location of tissue disposed between the first and second jaw members to the electrosurgical energy source, the location data including proximal, middle or distal tissue locations.

In aspects according to the present disclosure, the location data communicated to the electrosurgical energy source is used to determine a type of tissue treatment. In other aspects according to the present disclosure, the type of tissue treatment includes back scoring, sealing, poke and spread, and/or monopolar tissue treatment. In still other aspects according to the present disclosure, electrosurgical energy delivery to the tissue sealing plates is deactivated if the sensor determines the location of the tissue disposed between the first and second jaw members is improper for the type of tissue treatment.

Provided in accordance with aspects of the present disclosure is a surgical instrument including a housing having an elongated shaft extending distally therefrom and configured to support an end effector assembly at a distal end thereof. The end effector assembly includes first and second jaw members, each jaw member including a tissue sealing plate having at least proximal and distal segments disposed thereon. Each segment of each tissue sealing plate is adapted to independently connect to an electrosurgical energy source for delivery thereto upon activation thereof. Each segment of each tissue sealing plate communicates data relating to a location of tissue disposed between the first and second jaw members to the electrosurgical energy source. The electrosurgical energy source, in turn, uses the tissue location data to adjust one or more parameters associated with the delivery of electrosurgical energy to the tissue upon activation thereof.

In aspects according to the present disclosure, the one or more parameters associated with the delivery of electrosurgical energy to tissue includes power, current, voltage, duration, and/or pulse width. In other aspects according to the present disclosure, the location data communicated to the electrosurgical energy source is used to determine a type of tissue treatment. In still other aspects according to the present disclosure, the type of tissue treatment includes back scoring, sealing, poke and spread, and/or monopolar tissue treatment.

In aspects according to the present disclosure, electrosurgical energy delivery to the tissue sealing plates is deactivated if a segment of one or both tissue sealing plates determines the location of the tissue disposed between the first and second jaw members is improper for the type of tissue treatment.

In aspects according to the present disclosure, the segments are arranged in pairs on respective opposing tissue sealing plates and wherein the electrosurgical energy source is configured to emit a low energy signal across each pair of segments of the tissue sealing plates to induce a short therebetween in the presence of tissue, the electrosurgical energy source correlating the short between tissue sealing plates as data relating to the location of tissue disposed between the respective pair of segments and using the tissue location data to adjust one or more parameters associated with the delivery of electrosurgical energy to the tissue upon activation thereof. In other aspects according to the present disclosure, the location data communicated the electrosurgical energy source is used to determine a type of tissue treatment. In still other aspects according to the present disclosure, the type of tissue treatment includes back scoring, sealing, poke and spread, and/or monopolar tissue treatment.

Provided in accordance with aspects of the present disclosure is a system for treating tissue and includes a surgical forceps including a housing having an elongated shaft extending distally therefrom and configured to support an end effector assembly at a distal end thereof. The end effector assembly includes first and second jaw members, each jaw member including a tissue sealing plate disposed thereon. A generator is configured to supply electrosurgical energy to one or both of the tissue sealing plates upon activation thereof. A sensor is disposed on one or both of the tissue sealing plates of the first and second jaw members, the sensor configured to communicate data relating to a location of tissue disposed between the first and second jaw members to the electrosurgical energy source. The electrosurgical energy source, in turn, uses the tissue location data to adjust one or more parameters associated with the delivery of electrosurgical energy to the tissue upon activation thereof.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIGS. 1A-1D are views of an endoscopic electrosurgical forceps including a housing, an elongated shaft, an end effector and one embodiment of a sensor extending along the end effector;

FIGS. 2A and 2B are views of an open electrosurgical forceps including opposing shafts and an end effector assembly at a distal end thereof;

FIGS. 3A-3C are views of tissue disposed at various locations between the jaw members; and

FIG. 4 is a schematic illustration of a robotic surgical instrument provided in accordance with the present disclosure.

### DETAILED DESCRIPTION

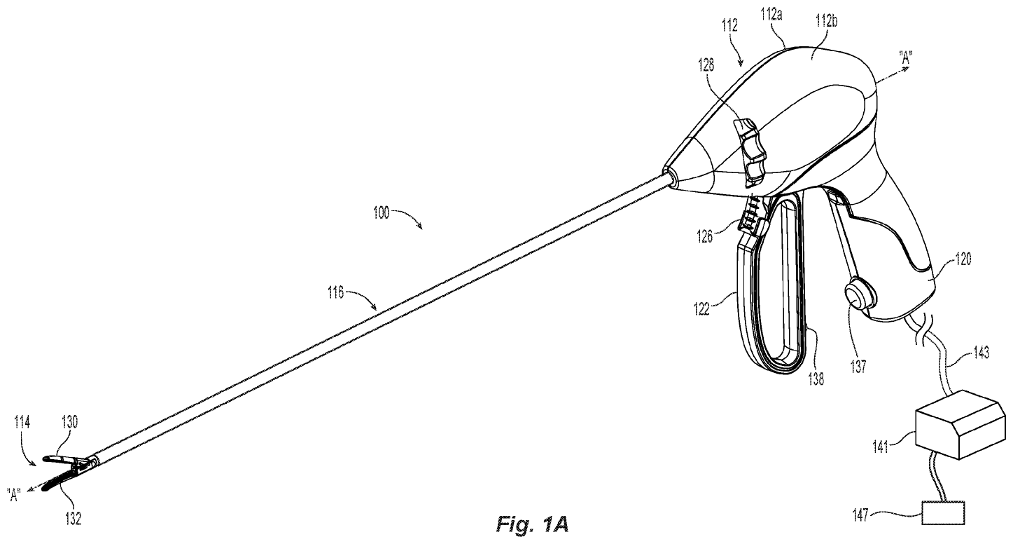

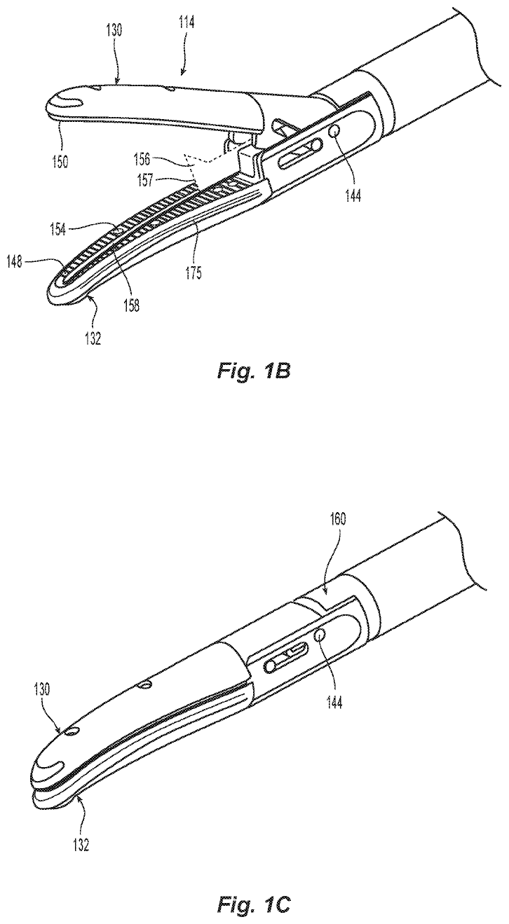

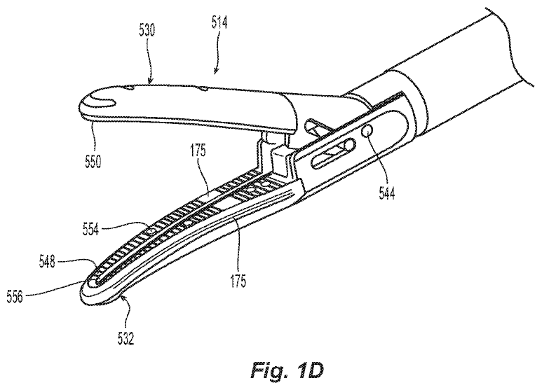

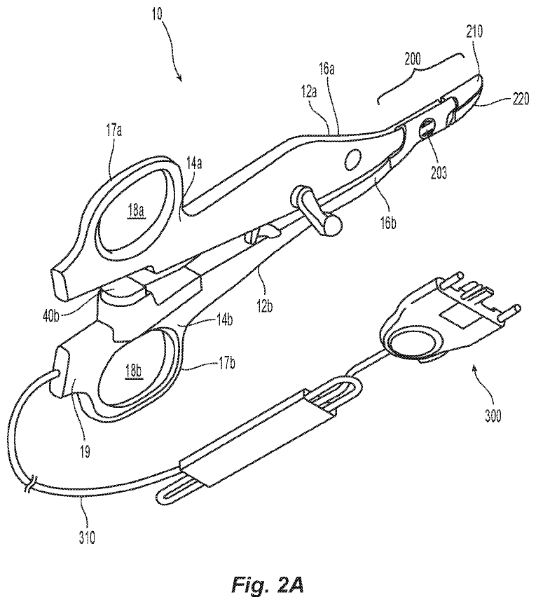

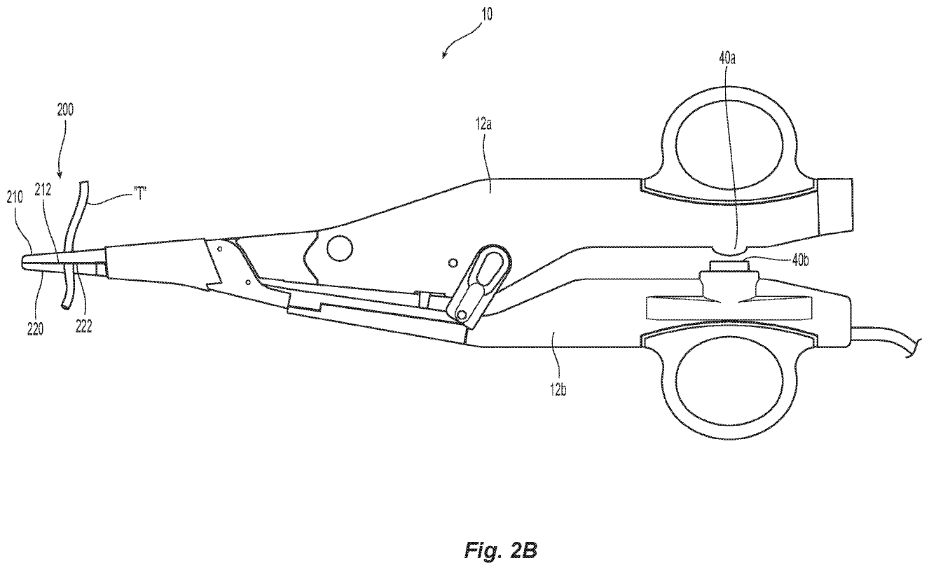

Referring initially to FIG. 1A, an endoscopic electrosurgical forceps 100 generally includes a housing 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well (See FIG. 2A). The housing 112 is constructed of a left housing half 112a and a right housing half 112b. The left and right designation of the housing halves 112a, 112b refer to the respective directions as perceived by an operator using the forceps 100. The housing halves 112a, 112b may be constructed of sturdy plastic, and may be joined to one another by adhesives, ultrasonic welding or other suitable assembly methods.

To mechanically control the end effector 114, the housing 112 supports a stationary handle 120, a movable handle 122, a trigger 126 and a rotation knob 128. The movable handle 122 is operable to move the end effector 114 between an open configuration wherein a pair of opposed jaw members 130, 132 are disposed in spaced relation relative to one another, and a closed or clamping configuration wherein the jaw members 130, 132 are closer together. Approximation of the movable handle 122 with the stationary handle 120 serves to move the end effector 114 to the closed configuration and separation of the movable handle 122 from the stationary handle 120 serves to move the end effector 114 to the open configuration. The trigger 126 is operable to extend and retract a knife blade 156 (FIG. 1B) through the end effector 114 when the end effector 114 is in the closed configuration. The rotation knob 128 serves to rotate the elongated shaft 116 and the end effector 114 about a longitudinal axis A-A extending through the forceps 100.

To electrically control the end effector 114, the stationary handle 120 supports a depressible button 137 thereon, which is operable by the user to initiate and terminate the delivery of electrosurgical energy to the end effector 114. The depressible button 137 is mechanically coupled to a switch (not shown) disposed within the stationary handle 120 which is in electrical communication with an electrosurgical generator 141 via suitable electrical wiring (not explicitly referenced) extending from the housing 112 through a cable 143 extending between the housing 112 and the electrosurgical generator 141. The generator 141 may include devices such as the LigaSure® Vessel Sealing Generator and the ForceTriad® Generator sold by Medtronic. The cable 143 may include a connector (not shown) thereon such that the forceps 100 may be selectively coupled electrically to the generator 141.

As mentioned above, the end effector 114 may be moved from the open configuration (FIG. 1B) wherein tissue (not shown) is received between the jaw members 130, 132, and the closed configuration (FIG. 1C), wherein the tissue is clamped and treated. The jaw members 130, 132 pivot about a pivot pin 144 (FIG. 1B) to move the end effector 114 to the closed configuration (FIG. 1C) of wherein sealing plates 148, 150 associated with respective jaw members 132, 130 provide a pressure to tissue grasped therebetween. In some embodiments, to provide an effective tissue seal, a pressure within a range between about 3 kg/cm² to about 16 kg/cm² and, desirably, within a working range of about 7 kg/cm² to about 13 kg/cm², may be applied to the tissue. Also, in the closed configuration, a separation or gap distance is maintained between the sealing plates 148, 150 by an array of stop members 154 (FIG. 1B) disposed on or adjacent the sealing plates 148, 150. The stop members 154 contact opposing surfaces on the opposing jaw member 130, 132 and prohibit further approximation of the sealing plates 148, 150. In some embodiments, to provide an effective tissue seal, an appropriate gap distance of about 0.001 inches to about 0.010 inches and, desirably, between about 0.002 inches to about 0.005 inches, may be provided. In some embodiments, the stop members 154 are constructed of a heat-resistant ceramic deposited onto the jaw members 130, 132. In other embodiments, the stop members 154 are constructed of an electrically non-conductive plastic molded onto the jaw members 130, 132, e.g., by a process such as overmolding or injection molding.

The upper and lower jaw members 130, 132 are electrically coupled to cable 143, and thus to the generator 141 (e.g., via respective suitable electrical wiring extending through the elongated shaft 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130. In some embodiments, the sealing plates 148 and 150 are electrically coupled to opposite terminals, e.g., positive or active (+) and negative or return (−) terminals associated with the generator 141. Thus, bipolar energy may be provided through the sealing plates 148 and 150 to tissue.

Alternatively, the sealing plates 148 and 150 may be configured to deliver monopolar energy to tissue. In a monopolar configuration, one or both sealing plates 148 and 150 deliver electrosurgical energy from an active terminal, e.g., (+), while a return pad (not shown) is placed generally on a patient and provides a return path to the opposite terminal, e.g., (−), of the generator 141. Each jaw member 130, 132 includes a jaw insert (not shown) and an insulator (not shown) that serves to electrically insulate the sealing plates 150, 148 from the jaw insert of the jaw members 130, 132, respectively.

Electrosurgical energy may be delivered to the tissue through the electrically conductive seal plates 148, 150 to effect a tissue seal. Once a tissue seal is established, the knife blade 156 having a sharpened distal edge 157 may be advanced through a knife channel 158 defined in one or both jaw members 130, 132 to transect the sealed tissue. Although the knife blade 156 is depicted in FIG. 1B as extending from the elongated shaft 116 when the end effector 114 is in an open configuration, in some embodiments, extension of the knife blade 156 into the knife channel 158 when the end effector 114 is in the open configuration may be prevented by one or more lockout features. An electrosurgical knife (FIG. 1D) may also be utilized to cut tissue. For example, U.S. Provisional Patent Application Ser. No. 63/056,113 filed Jul. 24, 2020 describes one such electrosurgical cutter and is incorporated by reference in its entirety herein.

Referring now to FIGS. 2A-2B, an open forceps 10 contemplated for use in connection with traditional open surgical procedures is shown. For the purposes herein, either an open instrument, e.g., forceps 10, or an endoscopic instrument (FIGS. 1A-1D) may be utilized in accordance with the present disclosure. Obviously, different electrical and mechanical connections and considerations apply to each particular type of instrument; however, the novel aspects with respect to the end effector assembly and its operating characteristics remain generally consistent with respect to both the open and endoscopic configurations.

With continued reference to FIGS. 2A-2B, forceps 10 includes two elongated shafts 12a and 12b, each having a proximal end 14a and 14b, and a distal end 16a and 16b, respectively. Forceps 10 further includes an end effector assembly 200 attached to distal ends 16a and 16b of shafts 12a and 12b, respectively. End effector assembly 200 includes a pair of opposing jaw members 210, 220 that are pivotably connected about a pivot 203. Each shaft 12a and 12b includes a handle 17a and 17b disposed at the proximal end 14a and 14b thereof. Each handle 17a and 17b defines a finger hole 18a and 18b therethrough for receiving a finger of the user. Finger holes 18a and 18b facilitate movement of the shaft members 12a and 12b relative to one another between a spaced-apart position and an approximated position, which, in turn, pivot jaw members 210, 220 from an open position, wherein the jaw members 210, 220 are disposed in spaced-apart relation relative to one another, to a closed position, wherein the jaw members 210, 220 cooperate to grasp tissue therebetween.

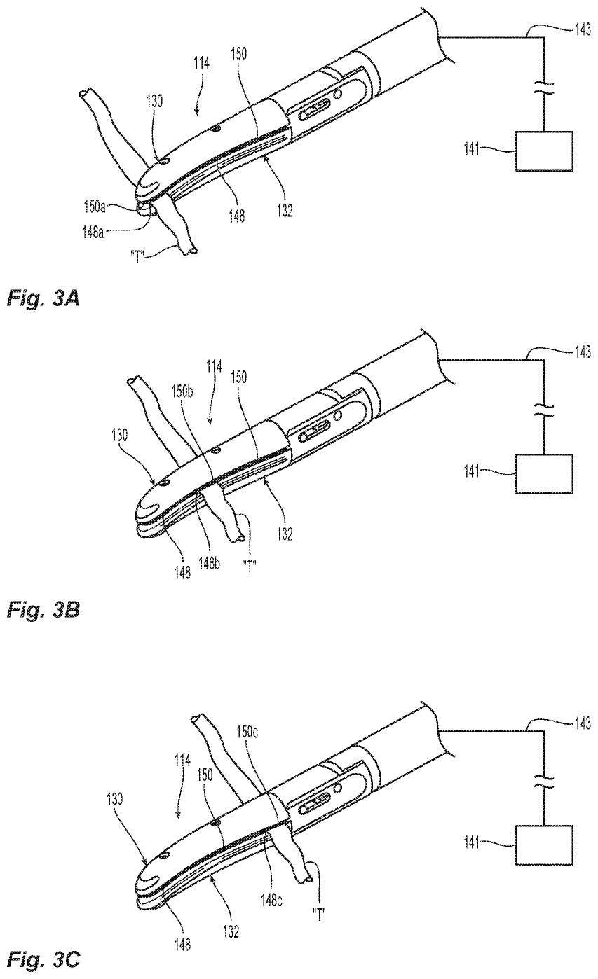

Continuing with reference to FIGS. 2A-2B, one of the shafts, e.g., shaft 12b, includes a proximal shaft connector 19 that is designed to connect the forceps 10 to a source of electrosurgical energy such as an electrosurgical generator 141 (FIGS. 1A, 3B). Proximal shaft connector 19 secures an electrosurgical cable 310 to forceps 10 such that the user may selectively apply electrosurgical energy to electrically-conductive plates 212, 222 (See FIG. 2B) of jaw members 210, 220, respectively.

More specifically, cable 310 includes a plurality of wires (not shown) extending therethrough that has sufficient length to extend through one of the shaft members, e.g., shaft member 12b, in order to provide electrical energy to the conductive plates 212, 222 of jaw members 210, 220, respectively, of end effector assembly 200, e.g., upon activation of activation switch 40b (See FIGS. 2A and 2B). Other types activation switches are also contemplated, e.g., finger switch, toggle switch, foot switch, etc. and may be configured for this purpose. Cable 310 operably connects to generator 141 via plug 300.

Activation switch 40b is disposed at proximal end 14b of shaft member 12b and extends therefrom towards shaft member 12a. A corresponding surface 40a (FIG. 2B) is defined along shaft member 12a toward proximal end 14a thereof and is configured to actuate activation switch 40b. More specifically, upon approximation of shaft members 12a, 12b, e.g., when jaw members 210, 220 are moved to the closed position, activation switch 40b is moved into contact with, or in close proximity of surface 40a. Upon further approximation of shaft members 12a, 12b, e.g., upon application of a pre-determined closure force to jaw members 210, 220, activation switch 40b is advanced further into surface 40a to depress activation switch 40b. Activation switch 40b controls the supply of electrosurgical energy to jaw members 210, 220 such that, upon depression of activation switch 40b, electrosurgical energy is supplied to conductive surface 212 and/or conductive surface 222 of jaw members 210, 220, respectively, to seal tissue grasped therebetween. The electrical energy may be energy supplied through a proprietary Ligasure® sealing algorithm owned by Medtronic. The switch 40b may be disposed on either shaft 12a, 12b.

Referring back to FIGS. 1A-1D once a vessel or tissue “T” or tissue is clamped between jaw members 130, 132 of end effector 114, the location of the tissue “T” on the tissue sealing plates 148, 150 is determined via any one of a number of sensors, e.g., sensor 175, that may be positioned thereon. Alternatively, and as discussed below, the location of tissue “T” on the tissue sealing plates 148, 150 between the jaw members 130, 132 may be determined utilizing sensors or various electrical characteristics of the tissue “T” in contact with the tissue sealing plates 148, 150, e.g., return electrode monitoring REM, impedance, low current sensing, etc. As such, one or both of the tissue sealing plates 148, 150 may need to be segmented to accurately reflect the location of the tissue “T” thereon. Segmenting the tissue sealing plates 148, 150 may help determine the size of the tissue “T” (or vessel) especially is the tissue “T” extends across multiple segmented areas.

As mentioned above, one or more sensors 175 may be disposed along one or both tissue sealing plates, e.g., plate 148. Sensor 175 is configured to provide feedback to the generator 141 regarding the location of the tissue “T” along one of the respective tissue sealing plate, e.g., tissue sealing plate 148, or, relative to both tissue sealing plates 148, 150. Location data may include proximal location, middle location and distal location. For example, if tissue “T” is disposed between the tissue sealing plates 148, 150 when tissue “T” is grasped therebetween, the sensor 175 provides location information back to the generator 141 which may be used to determine or adjust energy delivery parameters (power, current, voltage, duration, and/or pulse width) or the type of tissue treatment. If tissue is sensed at the distal location, more energy may be needed for a longer period of time to insure an accurate seal or treatment.

If two or more sensors 175 are provided on respective tissue sealing plates 148, 150, information relating to the location of tissue “T” may be communicated to the generator from the one or more sensors 175. For example, if a first sensor on jaw member 130 does not detect tissue “T” but a second sensor 175 on jaw member 132 detects tissue, the generator 141 can not only determine the location of the tissue “T” on tissue sealing plate 148 but also that the jaw members 130, 132 are open. In this instance, the generator 141 may be automatically configured to deliver energy in a monopolar fashion to tissue sealing plate 148 for back scoring tissue or to the cutting element 556 for open dissection. A footswitch 147 (FIG. 1A) may be utilized for this purpose.

In another example utilizing two or more sensors 175 along the tissue sealing plates 148, 150, the sensor(s) 175 may determine that no tissue “T” is disposed anywhere along either tissue sealing plate 148, 150, yet the surgeon is activating the generator 141 via a foot switch 147 (or hand switch if not an in-line activation device). In this instance, the generator 141 may recognize that the surgeon is attempting to supply energy to the tissue sealing plates 148, 150 with no tissue “T” disposed therebetween (or on one tissue sealing plate, e.g., 148, as discussed above) and may prevent activation as a safety feature.

In embodiments, the information provided by the one or more sensors 175 provided to the generator 141 may be inconsistent with the tissue treatment identified by the surgeon. As such, the generator 141 may remain deactivated until the surgeon corrects either the tissue location between the tissue sealing plate 148, 150, or selects a different tissue treatment option. Or, alternatively, the generator 141 may activate only a segment of the tissue sealing plate, e.g., distal segment 148a (as discussed below with respect to FIG. 3A) to allow the surgeon to poke and spread tissue “T”. The electrosurgical knife 556 (FIG. 1D) may also be activated when this condition is met and may be used for poke and spread depending on the configuration of the knife 556.

The forceps 10, 100 may be configured to recognize tissue “T” using other sensing techniques. For example, a low energy signal may be continuously sent across one or both tissue sealing plates 148, 150 and the generator 141 may be configured to sense a “short” condition. If no tissue is disposed between the tissue sealing plates 148, 150, the generator 141 will not sense a “short” condition and may prevent activation thereof. On the other hand, if tissue “T” is disposed between the tissue sealing plates 148, 150, the tissue “T” will cause a low energy “short” condition which is detected by the generator 141 freeing the generator 141 for activation via switch 137 (or footswitch 147). If using a monopolar electrical configuration, a return pad (not shown) may be utilized for short detection utilizing REM technology.

In this instance and in order to determine the location of the tissue “T” on the tissue sealing plates 148, 150, the tissue sealing plates 148, 150 may be segmented along the length thereof. Each segment, may be electrically connected to the generator 141 for determining a “short” condition with the presence of tissue “T”. Any number of segments may be utilized. For example, and as best shown in FIGS. 3A-3C, three pairs of different tissue sealing plate segments are utilized, e.g., distal segments 148a, 150a, middle segment 148b, 150b and proximal segment 148c, 150c.

FIG. 3A shows the pair of distal segments 148a, 150a with tissue “T” grasped therebetween. Once tissue “T” is sensed to be between the jaw members 148, 150 at segments 148a, 150a, a signal is communicated to the generator 141. Once communicated, the generator 141 may be configured to perform any number of functions prior to allowing activation via switch 137, 147 (or with respect to forceps 10, switch 40b). For example, if the tissue “T” is located at or proximate the distal ends of the jaw members 148, 150, the generator 141 may be configured to supply electrosurgical energy according to a different algorithm than when tissue “T” is disposed at a different location between tissue sealing plates 148, 150. Any number of energy and delivery parameters may be modified based on the location of the tissue “T”.

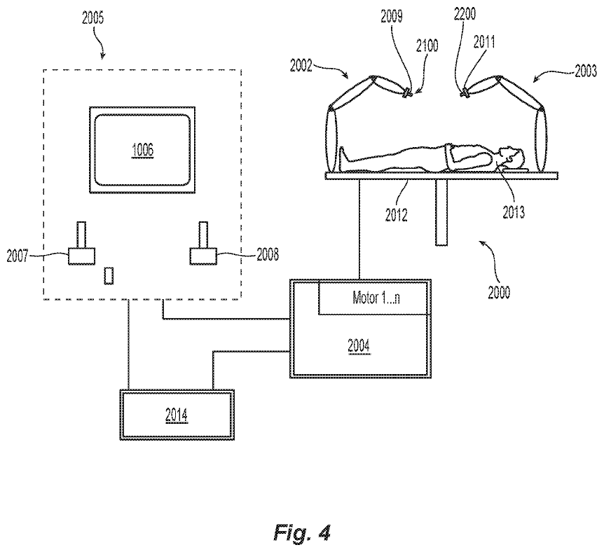

Moreover, the generator 141 may signal the user to apply additional force to the forceps 100, 10 based on tissue location, e.g., if tissue “T” is located between segments 148a, 150a, additional force may be necessary to insure an effective tissue seal. Or, if the jaw members 148, 150 are disposed on an end effector 114 of a robotic surgical instrument 1000 (FIG. 4), the generator may be configured to communicate with the robotic controller to apply more force between the tissue sealing plates 148, 150.

FIG. 3B shows the pair of middle segments 148b, 150b with tissue “T” grasped therebetween. Once tissue “T” is sensed to be between the jaw members 148, 150 at segments 148b, 150b, a signal is communicated to the generator 141. Once communicated and similar to FIG. 3A, the generator 141 may be configured to supply electrosurgical energy according to a specific algorithm wherein any number of energy and delivery parameters may be modified based on this location of the tissue “T”.

FIG. 3C shows the pair of proximal segments 148c, 150c with tissue “T” grasped therebetween. Once tissue “T” is sensed to be between the jaw members 148, 150 at segments 148c, 150c, a signal is communicated to the generator 141. Once communicated and similar to FIG. 3A, the generator 141 may be configured to supply electrosurgical energy according to a specific algorithm wherein any number of energy and delivery parameters may be modified based on this location of the tissue “T”. As mentioned above, locating the tissue “T” at a proximal-most location between the tissue sealing segments 148c, 150c may default the generator 141 to provide energy for back-scoring or dissecting tissue “T” if the jaw members 130, 132 are otherwise determined to be opened relative to one another.

If tissue “T” is disposed between more than one pair of sealing segments, e.g., segments 148b, 150b and 148c, 150c, the generator 141 may be configured to determine tissue size and adjust one or more energy parameters or tissue algorithms accordingly. For example, if the tissue “T” is located across multiple pairs of tissue sealing segments, the generator 141 may need to supply more energy than when tissue “T” is only located between one pair of sealing segments.

If tissue “T” is sensed between multiple sealing segments, e.g., segments 148a, 150a and 148c, 150c, and is not intended to be disposed between more than one pair of sealing segments, the generator 141 may be configured to deactivate as a safety measure to allow the surgeon to adjust the forceps 100, 10 or regrasp the intended tissue.

Any one of the above-mentioned techniques may be utilized to sense the tissue between the tissue sealing plates 148, 150 and adjust energy delivery accordingly or to select a tissue treatment.

Referring to FIG. 4, a robotic surgical instrument provided in accordance with the present disclosure is shown generally identified by reference numeral 2000. Aspects and features of robotic surgical instrument 2000 not germane to the understanding of the present disclosure are omitted to avoid obscuring the aspects and features of the present disclosure in unnecessary detail.

Robotic surgical instrument 2000 includes a plurality of robot arms 2002, 2003; a control device 2004; and an operating console 2005 coupled with control device 2004. Operating console 2005 may include a display device 2006, which may be set up in particular to display three-dimensional images; and manual input devices 2007, 2008, by means of which a surgeon may be able to telemanipulate robot arms 2002, 2003 in a first operating mode. Robotic surgical instrument 2000 may be configured for use on a patient 2013 lying on a patient table 2012 to be treated in a minimally invasive manner. Robotic surgical instrument 2000 may further include a database 21014, in particular coupled to control device 2004, in which are stored, for example, pre-operative data from patient 2013 and/or anatomical atlases.

Each of the robot arms 2002, 2003 may include a plurality of members, which are connected through joints, and an attaching device 2009, 2011, to which may be attached, for example, an end effector assembly 2100, 2200, respectively. End effector assembly 2100 is similar to end effector assembly 100 (FIG. 4), although other suitable end effector assemblies for coupling to attaching device 2009 are also contemplated. End effector assembly 2200 may be any end effector assembly, e.g., an endoscopic camera, other surgical tool, etc. Robot arms 2002, 2003 and end effector assemblies 2100, 2200 may be driven by electric drives, e.g., motors, that are connected to control device 2004. Control device 2004 (e.g., a computer) may be configured to activate the motors, in particular by means of a computer program, in such a way that robot arms 2002, 2003, their attaching devices 2009, 2011, and end effector assemblies 2100, 2200 execute a desired movement and/or function according to a corresponding input from manual input devices 2007, 2008, respectively. Control device 2004 may also be configured in such a way that it regulates the movement of robot arms 2002, 2003 and/or of the motors.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

1. A surgical instrument, comprising:
a housing;
an elongated shaft extending distally from the housing and configured to support an end effector assembly at a distal end thereof, the end effector assembly including first and second jaw members, each jaw member including a tissue sealing plate disposed thereon adapted to connect to an electrosurgical energy source for delivery thereto upon activation thereof; and
a sensor disposed on at least one of the tissue sealing plates of the first and second jaw members, the sensor configured to communicate data relating to a location of tissue disposed between the first and second jaw members to the electrosurgical energy source, the electrosurgical energy source, in turn, using the tissue location data to adjust one or more parameters associated with the delivery of electrosurgical energy to the tissue upon activation thereof.

2. The surgical instrument according to claim 1, wherein the one or more parameters associated with the delivery of electrosurgical energy to tissue includes at least one of power, current, voltage, duration, or pulse width.

3. The surgical instrument according to claim 1, wherein the surgical instrument includes a plurality of sensors disposed along the at a least one tissue sealing plate, the plurality of sensors communicating data relating to the location of tissue disposed between the first and second jaw members to the electrosurgical energy source, the location data including proximal, middle or distal tissue locations.

4. The surgical instrument according to claim 3, wherein the location data communicated to the electrosurgical energy source is used to determine a type of tissue treatment.

5. The surgical instrument according to claim 4, wherein the type of tissue treatment includes at least one of back scoring, sealing, poke and spread, or monopolar tissue treatment.

6. The surgical instrument according to claim 4, wherein electrosurgical energy delivery to the tissue sealing plates is deactivated if the sensor determines the location of the tissue disposed between the first and second jaw members is improper for the type of tissue treatment.

7. A surgical instrument, comprising:
a housing;
an elongated shaft extending distally from the housing and configured to support an end effector assembly at a distal end thereof, the end effector assembly including first and second jaw members, each jaw member including a tissue sealing plate having at least proximal and distal segments disposed thereon, each segment of each tissue sealing plate adapted to independently connect to an electrosurgical energy source for delivery thereto upon activation thereof, each segment of each tissue sealing plate communicating data relating to a location of tissue disposed between the first and second jaw members to the electrosurgical energy source, the electrosurgical energy source, in turn, using the tissue location data to adjust one or more parameters associated with the delivery of electrosurgical energy to the tissue upon activation thereof.

8. The surgical instrument according to claim 7, wherein the one or more parameters associated with the delivery of electrosurgical energy to tissue includes at least one of power, current, voltage, duration, or pulse width.

9. The surgical instrument according to claim 7, wherein the location data communicated to the electrosurgical energy source is used to determine a type of tissue treatment.

10. The surgical instrument according to claim 9, wherein the type of tissue treatment includes at least one of back scoring, sealing, poke and spread, or monopolar tissue treatment.

11. The surgical instrument according to claim 9, wherein electrosurgical energy delivery to the tissue sealing plates is deactivated if a segment of one or both tissue sealing plates determines the location of the tissue disposed between the first and second jaw members is improper for the type of tissue treatment.

12. The surgical instrument according to claim 7, wherein the segments are arranged in pairs on respective opposing tissue sealing plates and wherein the electrosurgical energy source is configured to emit a low energy signal across each pair of segments of the tissue sealing plates to induce current flow or a short therebetween in the presence of tissue, the electrosurgical energy source correlating the short between tissue sealing plates as data relating to the location of tissue disposed between the respective pair of segments and using the tissue location data to adjust one or more parameters associated with the delivery of electrosurgical energy to the tissue upon activation thereof.

13. The surgical instrument according to claim 12, wherein the location data communicated to the electrosurgical energy source is used to determine a type of tissue treatment.

14. The surgical instrument according to claim 13, wherein the type of tissue treatment includes at least one of back scoring, sealing, poke and spread, or monopolar tissue treatment.

15. A system for treating tissue, comprising:
a surgical forceps including a housing having an elongated shaft extending distally therefrom and configured to support an end effector assembly at a distal end thereof, the end effector assembly including first and second jaw members, each jaw member including a tissue sealing plate disposed thereon;
a generator configured to supply electrosurgical energy to at least one of the tissue sealing plates upon activation thereof; and
a sensor disposed on at least one of the tissue sealing plates of the first and second jaw members, the sensor configured to communicate data relating to a location of tissue disposed between the first and second jaw members to the electrosurgical energy source, the electrosurgical energy source, in turn, using the tissue location data to adjust one or more parameters associated with the delivery of electrosurgical energy to the tissue upon activation thereof.
